(19) United States
(12) Patent Application Publication
(10) Pub. No.: US 2022/0226034 A1
(43) Pub. Date: Jul. 21, 2022

(54) OCCLUSION DETECTION USING BLOOD FLOW MEASUREMENT

(71) Applicant: Boston Scientific Scimed Inc, Maple Grove, MN (US)

(72) Inventor: El Yacine Alex Smail, Vaudreuil-Dorion (CA)

(21) Appl. No.: 17/562,525

(22) Filed: Dec. 27, 2021

Related U.S. Application Data
(60) Provisional application No. 63/130,984, filed on Dec. 28, 2020.

(51) Int. Cl. A61B 18/02

(52) U.S. Cl. CPC A61B 18/02; A61B 2018/00863

(57) ABSTRACT

A control system for a cryogenic ablation system. The control system including an extracorporeal ultrasound sensor and a controller. The extracorporeal ultrasound sensor is configured to detect blood flow in a vein and generate an output signal indicative of blood flow velocity of the blood flow in the vein, and the controller is configured to receive the output signal and determine whether the vein has been occluded by a cryoablation balloon catheter.

### CROSS REFERENCE TO RELATED APPLICATION

This application claims priority to Provisional Application No. 63/130,984, filed Dec. 28, 2020, which is herein incorporated by reference in its entirety.

### TECHNICAL FIELD

The present invention relates to medical devices and methods for treating cardiac arrhythmias. More specifically, the invention relates to devices and methods for applying cryotherapy to cardiac tissues.

### BACKGROUND

Cardiac arrhythmias involve an abnormality in the electrical conduction of the heart and are a leading cause of stroke, heart disease, and sudden cardiac death. Treatment options for patients with arrhythmias include medications and/or the use of medical devices, which can include implantable devices and/or catheter ablation of cardiac tissue, to name a few. Catheter ablation involves delivering ablative energy to tissue inside the heart to block aberrant electrical activity from depolarizing heart muscle cells out of synchrony with the heart's normal conduction pattern. The procedure is performed by positioning the tip of an energy delivery catheter adjacent to diseased or targeted tissue in the heart. Typically, the energy delivery component of the system is at or near the most distal portion of the catheter, i.e., farthest from the user or operator, and often at the tip of the catheter.

Various forms of energy can be used to ablate diseased heart tissue. These can include radio frequency (RF), cryogenics, ultrasound, and laser energy, to name a few. During a cryoablation procedure, with the aid of a guide wire, the distal tip of the catheter is positioned adjacent to targeted cardiac tissue, at which time energy is delivered to create tissue necrosis, rendering the ablated tissue incapable of conducting electrical signals.

Atrial fibrillation (AF) is a common arrhythmia treated using catheter ablation. One AF treatment strategy involves isolating the pulmonary veins from the left atrial chamber. A particularly useful technique known as catheter balloon cryotherapy or cryoablation can be employed to treat AF. During balloon cryoablation procedures, a balloon on a balloon catheter is positioned within the ostium of the pulmonary vein to be treated and inflated to intimately contact the surrounding tissue and occlude the pulmonary vein. Traditionally, the operator will inflate the balloon with no cooling fluid provided therein, and verify occlusion using methods such as injection contrast and fluoroscopy prior to delivering the ablative, i.e., cryogenic, energy. However, using contrast agent injection to ensure pulmonary vein occlusion exposes the patient and medical personnel to radiation and requires the use of lead aprons.

There is a continuing need for improved systems and methods for verifying and ensuring pulmonary vein occlusion in pulmonary vein isolation procedures.

### SUMMARY

Example 1 is a control system for a cryogenic ablation system. The control system includes an extracorporeal ultrasound sensor and a controller. The extracorporeal ultrasound sensor is configured to detect blood flow in a vein and generate an output signal indicative of blood flow velocity of the blood flow in the vein, and the controller is configured to receive the output signal and determine whether the vein has been occluded by a cryoablation balloon catheter.

In Example 2, the system of Example 1, wherein the controller is configured to determine changes in the blood flow velocity, determine average changes in the changes in the blood flow velocity during multiple time periods, and compare one or more of the average changes to a stability threshold value to determine whether the vein has been occluded.

In Example 3, the system of any one of Examples 1 and 2, wherein the controller determines an overall change in the blood flow velocity and compares the overall change in the blood flow velocity to an overall change threshold value to determine whether the vein has been occluded.

In Example 4, the system of any one of Examples 1-3, wherein the controller is an ultrasound controller and the extracorporeal ultrasound sensor is coupled to the ultrasound controller and configured to transmit and receive ultrasound frequencies through patient tissues, wherein the ultrasound controller is configured to determine the blood flow velocity.

In Example 5, the system of any one of Examples 1-4, wherein the controller is configured to determine the blood flow velocity based on a doppler frequency shift in the blood flow.

In Example 6, the system of any one of Examples 1-5, wherein the controller prevents ablation from proceeding until the controller has determined that the vein has been occluded.

In Example 7, the system of any one of Examples 1-6, wherein the controller provides one or more of an audio and a visual alarm in response to loss of occlusion of the vein.

Example 8 is a cryogenic ablation system for determining whether a pulmonary vein has been occluded. The system including a cryoablation balloon catheter, an extracorporeal ultrasound sensor, and a controller. The cryoablation balloon catheter configured to be inserted into an ostium of the pulmonary vein. The extracorporeal ultrasound sensor configured to detect blood flow in the pulmonary vein before, during, and after inflation of the cryoablation balloon catheter in the ostium of the pulmonary vein and to generate output signals indicative of blood flow velocity of the blood flow in the pulmonary vein. The controller coupled to the extracorporeal ultrasound sensor and configured to receive the output signals and determine blood flow velocity values to determine whether the pulmonary vein has been occluded by the cryoablation balloon catheter.

In Example 9, the system of Example 8, wherein the controller is configured to inflate the cryoablation balloon catheter in response to manually pushing an inflate control button.

In Example 10, the system of any one of Examples 8 and 9, wherein the controller determines at least one of average changes in changes of the blood flow velocity values during multiple time periods and an overall change in the blood flow velocity values from prior to inflation of the cryoablation balloon catheter to after inflation of the cryoablation balloon catheter.

In Example 11, the system of any one of Examples 8-10, further comprising a display, wherein the controller is configured to visually indicate on the display an occlusion level of the pulmonary vein.

In Example 12, the system of any one of Examples 8-11, further comprising a display, wherein the controller is configured to visually indicate on the display that ablation may proceed.

Example 13 is a method of determining a level of occlusion of a vein. The method including: inserting a cryoablation balloon catheter into an ostium of the vein; obtaining, by an ultrasound system, at least one blood flow velocity measurement of blood flow in the vein prior to inflation of the cryoablation balloon catheter; inflating the cryoablation balloon catheter in the ostium of the vein; obtaining, by the ultrasound system, multiple blood flow velocity measurements of the blood flow in the vein during inflation of the cryoablation balloon catheter; and determining, by a controller coupled to the ultrasound system and using the at least one blood flow velocity measurement and the multiple blood flow velocity measurements, the level of occlusion of the vein.

In Example 14, the method of Example 13, wherein determining the level of occlusion of the vein includes determining changes of blood flow velocity in the vein, determining average changes in the changes of the blood flow velocity during multiple time periods, and comparing one or more of the average changes to a threshold value.

In Example 15, the method of any one of Examples 13 and 14, wherein determining the level of occlusion of the vein includes determining an overall change of blood flow velocity in the vein from prior to inflation of the cryoablation balloon catheter to after inflation of the cryoablation balloon catheter and comparing the overall change of the blood flow velocity in the vein to a threshold value.

Example 16 is a control system for a cryogenic ablation system. The control system including an extracorporeal ultrasound sensor and a controller. The extracorporeal ultrasound sensor configured to detect blood flow in a vein and generate an output signal indicative of blood flow velocity of the blood flow in the vein, and the controller configured to receive the output signal and determine whether the vein has been occluded by a cryoablation balloon catheter.

In Example 17, the system of Example 16, wherein the controller is configured to determine changes in the blood flow velocity and to determine average changes in the changes in the blood flow velocity during multiple time periods to determine whether the vein has been occluded.

In Example 18, the system of Example 17, wherein the controller compares one or more of the average changes to a stability threshold value to determine whether the vein has been occluded.

In Example 19, the system of Example 16, wherein the controller determines an overall change in the blood flow velocity to determine whether the vein has been occluded.

In Example 20, the system of Example 19, wherein the controller compares the overall change in the blood flow velocity to an overall change threshold value to determine whether the vein has been occluded.

In Example 21, the system of Example 16, wherein the vein is a pulmonary vein.

In Example 22, the system of Example 16, wherein the controller is an ultrasound controller and the extracorporeal ultrasound sensor is coupled to the ultrasound controller and configured to transmit and receive ultrasound frequencies through patient tissues, wherein the ultrasound controller is configured to determine the blood flow velocity.

In Example 23, the system of Example 16, wherein the controller is configured to determine the blood flow velocity based on a doppler frequency shift in the blood flow.

In Example 24, the system of Example 16, wherein the controller prevents ablation from proceeding until the controller has determined that the vein has been occluded.

In Example 25, the system of Example 16, wherein the controller provides one or more of an audio and a visual alarm in response to loss of occlusion of the vein.

Example 26 is a cryogenic ablation system for determining whether a pulmonary vein has been occluded. The system including a cryoablation balloon catheter, an extracorporeal ultrasound sensor, and a controller. The cryoablation balloon catheter configured to be inserted into an ostium of the pulmonary vein. The extracorporeal ultrasound sensor configured to detect blood flow in the pulmonary vein before, during, and after inflation of the cryoablation balloon catheter in the ostium of the pulmonary vein and to generate output signals indicative of blood flow velocity of the blood flow in the pulmonary vein. The controller coupled to the extracorporeal ultrasound sensor and configured to receive the output signals and determine blood flow velocity values to determine whether the pulmonary vein has been occluded by the cryoablation balloon catheter.

In Example 27, the system of Example 26, wherein the controller is configured to inflate the cryoablation balloon catheter in response to manually pushing an inflate control button.

In Example 28, the system of Example 26, wherein the controller determines at least one of average changes in changes of the blood flow velocity values during multiple time periods and an overall change in the blood flow velocity values from prior to inflation of the cryoablation balloon catheter to after inflation of the cryoablation balloon catheter.

In Example 29, the system of Example 26, further comprising a display, wherein the controller is configured to visually indicate on the display an occlusion level of the pulmonary vein.

In Example 30, the system of Example 26, further comprising a display, wherein the controller is configured to visually indicate on the display that ablation may proceed.

Example 31 is a method of determining a level of occlusion of a vein. The method including: inserting a cryoablation balloon catheter into an ostium of the vein; obtaining, by an ultrasound system, at least one blood flow velocity measurement of blood flow in the vein prior to inflation of the cryoablation balloon catheter; inflating the cryoablation balloon catheter in the ostium of the vein; obtaining, by the ultrasound system, multiple blood flow velocity measurements of the blood flow in the vein during inflation of the cryoablation balloon catheter; and determining, by a controller coupled to the ultrasound system and using the at least one blood flow velocity measurement and the multiple blood flow velocity measurements, the level of occlusion of the vein.

In Example 32, the method of Example 31, wherein determining the level of occlusion of the vein includes determining changes of blood flow velocity in the vein and determining average changes in the changes of the blood flow velocity during multiple time periods.

In Example 33, the method of Example 32, wherein determining the level of occlusion of the vein includes comparing one or more of the average changes to a threshold value.

In Example 34, the method of Example 31, wherein determining the level of occlusion of the vein includes determining an overall change of blood flow velocity in the vein from prior to inflation of the cryoablation balloon catheter to after inflation of the cryoablation balloon catheter.

In Example 35, the method of Example 34, wherein determining the level of occlusion of the vein includes comparing the overall change of the blood flow velocity in the vein to a threshold value.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a simplified schematic side view illustration of a patient and a cryogenic balloon catheter system, according to embodiments of the disclosure;

FIG. 2A is a simplified schematic view illustration of a portion of the patient and a portion of the cryogenic balloon catheter system with an expandable balloon that is partially inflated or deflated in the pulmonary vein and blood flow in the pulmonary vein and past the expandable balloon, according to embodiments of the disclosure.

FIG. 2B is a simplified schematic view illustration of a portion of the patient and a portion of the cryogenic balloon catheter system with the expandable balloon inflated to occlude the pulmonary vein, such that blood flow past the expandable balloon is prevented, according to embodiments of the disclosure.

FIG. 3A is a diagram illustrating the graphical display including an occlusion level indicator prior to reaching a satisfactory level of occlusion for ablation to proceed, according to embodiments of the disclosure.

FIG. 3B is a diagram illustrating the graphical display including the occlusion level indicator and an ablation button, after reaching a level of occlusion where ablation may proceed, according to embodiments of the disclosure.

FIG. 4 is a flow chart diagram illustrating a method of determining a level of occlusion of a vein and/or whether the vein has been occluded, according to embodiments of the disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

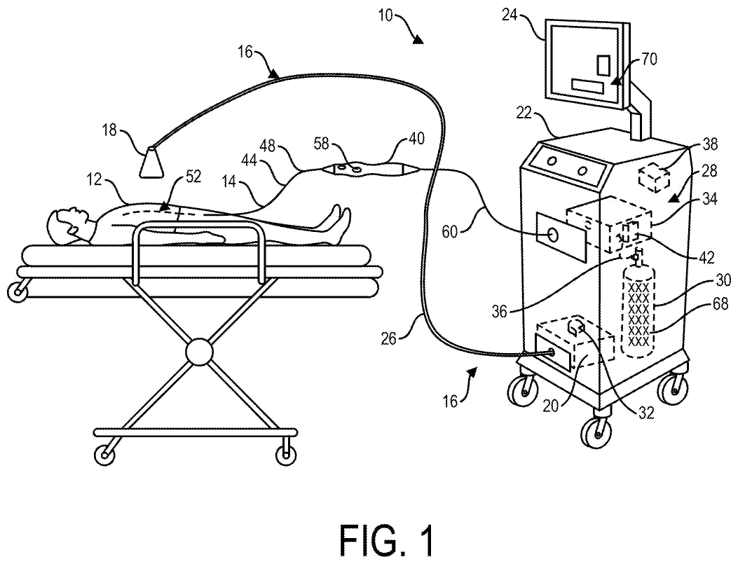

FIG. 1 is a simplified schematic side view illustration of a cryogenic balloon catheter system 10 for use with a patient 12, which can be a human being or an animal, according to embodiments of the disclosure. Although the design of the cryogenic balloon catheter system 10 can be varied depending on the particular clinical needs of the patient 12, in the illustrated example the cryogenic balloon catheter system 10 includes one or more of a balloon catheter 14, an extracorporeal ultrasound system 16, a control console 22, a graphical display 24, and a fluid control system 28. Also, although FIG. 1 illustrates the structures of the cryogenic balloon catheter system 10 in a specific position, sequence and/or order, in other examples, these structures can be in different positions, sequences, and/or order than that illustrated in FIG. 1. In addition, the cryogenic balloon catheter system 10 can include fewer or additional components than those specifically illustrated and described herein.

The cryogenic balloon catheter system 10 is configured to improve vein occlusion in vein isolation procedures, such as in pulmonary vein isolation procedures, by improving verification of vein occlusion in these procedures. In the cryogenic balloon catheter system 10, the extracorporeal ultrasound system 16 is used to monitor blood flow through a vein of interest to determine whether blood flow through the vein has been stopped or substantially stopped, such that the vein has been occluded and ablation can proceed.

The extracorporeal ultrasound system 16 includes an extracorporeal ultrasound transducer or sensor 18 and an ultrasound control system 20 that is illustrated in phantom and disposed within the control console 22. The extracorporeal ultrasound transducer 18 is electrically and communicatively coupled to the ultrasound control system 20 by conductive path 26. The ultrasound control system 20 includes a controller 32, such as a microprocessor, and memory storing code that is executable by the controller 32 to perform functions of the cryogenic balloon catheter system 10. Also, in embodiments, the control console 22 includes a controller 34, such as a microprocessor, and memory storing code that is executable by the controller 34 to perform functions of the cryogenic balloon catheter system 10.

In embodiments, the ultrasound control system 20 determines the velocity of blood flowing through the vein of interest, such as a pulmonary vein, from signals received by the ultrasound control system 20 from the extracorporeal ultrasound transducer 18. The extracorporeal ultrasound transducer or sensor 18 is configured to detect blood flow through the vein and generate an output signal indicative of blood flow velocity of the blood flow through the vein. The controller 32 of the ultrasound control system 20 is configured to receive the output signal and determine the velocity of the blood flow in the vein. Using these velocity measurements, the controller 32 of the ultrasound control system 20 or the controller 38 of the control console 22 determines whether the vein has been occluded, such as by a cryoablation balloon catheter. In embodiments, the controller 32 of the ultrasound control system 20 or the controller 38 of the control console 22 determines the degree or level of occlusion of the vein and whether ablation may proceed. Also, in embodiments, the controller 38 of the control console 22 can be configured to receive the output signal and determine the velocity of the blood flow in the vein.

The ultrasound control system 20 and/or the control console 22 are communicatively coupled to the graphical display 24, which can include a graphical user interface (GUI), to display at least one of whether the vein has been occluded, the degree or level of occlusion of the vein, and whether ablation may proceed.

In further description of the cryogenic balloon catheter system 10, the balloon catheter 14 includes a handle assembly 40, and a shaft 44 having a proximal end portion 48 connected to the handle assembly 40, and a distal end portion 52, shown disposed within the patient 12. As will be appreciated, the handle assembly 40 can include various components, such as a control element 58, that the user can manipulate to operate the balloon catheter 14. Also, an umbilical 60 operatively connects the handle assembly 40 and the active components of the balloon catheter 14 to the control console 22. In embodiments, the system 10 may include additional components or alternative approaches to operatively connect the balloon catheter 14 to the control console 22.

The fluid control system 28 includes a fluid source 30 and a fluid control arrangement 34, which are illustrated in phantom and disposed within the control console 22. In embodiments, the fluid control system 28 can include various conduits, valves and instrumentation configured to supply and withdraw a fluid to the active elements on the balloon catheter 14. The fluid source 30 is operably connected to the fluid control arrangement 34 by a conduit 36, which may be in the form of a hose or tubing, configured to transfer fluid contained within the fluid source 30 to components making up the fluid control arrangement 34.

The fluid control system 28 includes a controller 42, such as a microprocessor, and memory storing code that is executable by the controller 42 to perform the functions of the fluid control system 28. In embodiments, the fluid control system 28 is configured to monitor and control various processes of the ablation procedures performed with the cryogenic balloon catheter system 10. More specifically, the fluid control system 28 can monitor and control release and/or retrieval of a cooling fluid 68, e.g., a cryogenic fluid, shown schematically contained within the fluid source 30, to the balloon catheter 14, e.g., via fluid injection and fluid exhaust lines (not shown), but which may be disposed within the umbilical 60. The fluid control system 28 can also control various structures that are responsible for maintaining and/or adjusting a flow rate and/or pressure of the cryogenic fluid 68 that is released to the balloon catheter 14 during the cryoablation procedure. In embodiments, the cryogenic balloon catheter system 10 delivers ablative energy in the form of cryogenic fluid 68 to cardiac tissue of the patient 12 to create tissue necrosis, rendering the ablated tissue incapable of conducting electrical signals. Additionally, in embodiments, the fluid control system 28 can control activation and/or deactivation of one or more other processes of the balloon catheter 14.

Further, or in the alternative, the fluid control system 28 can receive data and/or other information, hereinafter sometimes referred to as sensor output, from various structures within the cryogenic balloon catheter system 10. In some embodiments, the fluid control system 28 can receive, monitor, assimilate and/or integrate the sensor output, and/or any other data or information received from any structure within the cryogenic balloon catheter system 10 in order to control the operation of the balloon catheter 14. Also, in embodiments, the fluid control system 28 can initiate and/or terminate the flow of cryogenic fluid 68 to the balloon catheter 14 based on the sensor output.

As shown in FIG. 1, in embodiments, the fluid control system 28 can be positioned substantially within the control console 22. Alternatively, at least a portion of the fluid control system 28 can be positioned in one or more other locations within the cryogenic balloon catheter system 10, e.g., within the handle assembly 40.

The fluid source 16 contains the cryogenic fluid 68, which is delivered to and from the balloon catheter 14 with or without input from the fluid control system 28 during a cryoablation procedure. Once the ablation procedure has initiated, the cryogenic fluid 68 can be delivered and the resulting gas, after a phase change, can be retrieved from the balloon catheter 14, and can either be vented or otherwise discarded as exhaust. Additionally, the type of cryogenic fluid 68 that is used during the cryoablation procedure can vary. In some embodiments, the cryogenic fluid 68 can include liquid nitrous oxide. However, any other suitable cryogenic fluid 68 can be used. For example, in some embodiments, the cryogenic fluid 68 can include liquid nitrogen.

The design of the balloon catheter 14 can be varied to suit the specific design requirements of the cryogenic balloon catheter system 10. As shown, the balloon catheter 14 is inserted into the body of the patient 12 during the cryoablation procedure. The handle assembly 40 can be handled and used by the operator to operate, position and control the balloon catheter 14. The design and specific features of the handle assembly 40 can vary to suit the design requirements of the cryogenic balloon catheter system 10. In embodiments, the handle assembly 40 is separate from, but in electrical and/or fluid communication with the fluid control system 28, the fluid source 16, and the graphical display 24. In some embodiments, the handle assembly 40 can integrate and/or include at least a portion of the fluid control system 28 within an interior of the handle assembly 40. Also, it is understood that the handle assembly 40 can include fewer or additional components than those specifically illustrated and described herein and, in some embodiments, the handle assembly 40 can include circuitry (not shown in FIG. 1) that can include at least a portion of the fluid control system 28. For example, the circuitry can transmit electrical signals such as the sensor output, or otherwise provide data to the fluid control system 28. In embodiments, the circuitry can include a printed circuit board having one or more integrated circuits and/or other circuits. In some embodiments, the handle assembly 40 can be used by the operator to initiate and/or terminate the cryoablation process, e.g., to start the flow of the cryogenic fluid 68 to the balloon catheter 14 in order to ablate certain targeted heart tissue of the patient 12.

In embodiments, the control console 22 includes at least a portion of the extracorporeal ultrasound system 16, the fluid control system 28, the fluid source 30, and the graphical display 24. Also, in embodiments, the control console 22 can contain additional structures not shown or described herein or the control console 22 may not include structures that are illustrated within the control console 22 in FIG. 1. For example, in embodiments, the control console 22 does not include the graphical display 24.

During cryoablation procedures, the balloon catheter 14 and the control console 22 are operatively connected to allow the flow of cryogenic fluid 68 from the control console 22 to the balloon catheter 14 and back to the control console 22. Generally, during the application of ablative energy, the cryogenic fluid 68 flows in a liquid phase to the balloon catheter 14, where the cryogenic fluid 68 undergoes a phase change and returns to the control console 22 as exhaust in a gaseous phase.

In embodiments, the graphical display 24 is electrically connected to one or more of the fluid control system 28, the control console 22, and the ultrasound control system 20. Additionally, the graphical display 24 provides the operator of the cryogenic balloon catheter system 10 with information that can be used before, during, and after the cryoablation procedure. For example, the graphical display 24 can provide the operator with information based on the sensor output, and any other relevant information that can be used before, during, and after the cryoablation procedure. The specifics of the graphical display 24 can vary depending upon the design requirements of the cryogenic balloon catheter system 10, and the specific needs, specifications, and/or desires of the operator.

In embodiments, the graphical display 24 can provide static visual data and/or information to the operator via various frames or other representations 70. In addition, the graphical display 24 can provide dynamic visual data and/or information to the operator, such as video data or any other data that changes over time, e.g., during an ablation procedure. Further, in embodiments, the graphical display 24 can include one or more colors, different display sizes, varying brightness, etc., that may act as alerts to the operator. Also, in embodiments, the graphical display 24 can provide audio data or information to the operator. In embodiments, the graphical display 24 includes a GUI.

In operation of the cryogenic balloon catheter system 10, the balloon catheter 14 is inserted into the vein of interest, such as into an ostium of the vein of interest, in the patient 12 and the extracorporeal ultrasound system 16 is used to obtain one or more blood flow velocity measurements or values of the blood flow in the vein. Next, the cryoablation balloon catheter 14 is inflated to occlude the vein, reducing and preventing blood flow in or through the vein, and the extracorporeal ultrasound system 16 is used again to obtain more blood flow velocity values of the blood flow through the vein during inflation of the balloon catheter 14 and after inflation of the balloon catheter 14. Based on the obtained blood flow velocity values, the cryogenic balloon catheter system 10 determines one or more of whether the vein is occluded, the degree or level of occlusion of the vein, and whether the cryogenic balloon catheter system 10 may proceed with ablation. In embodiments, if the level of occlusion of the vein does not meet a threshold level of occlusion, the cryogenic balloon catheter system 10 locks the system 10, preventing the system 10 from providing ablation. In embodiments, if occlusion has been achieved and then lost during a vein isolation procedure, the cryogenic balloon catheter system 10, such as the control console 22, sets off an alarm to alert the user, where the alarm may be a visual and/or audio alarm provided via the graphical display 24.

In embodiments, each of the blood flow velocity measurements is determined by the control console 22 or the ultrasound control system 20. In embodiments, the velocity V of the blood flowing in or through the vein is determined using a doppler ultrasound measurement technique and the following Formula 1:

$${f}_{d} = \frac{2 ( {f}_{c} \times cos θ \times V )}{c} \qquad Formula 1$$

where, fd is the doppler frequency, V is the velocity of the blood in the vein, theta is the angle between the transducer and the vein, c is the speed of sound moving through soft tissues, which is approximately 1.5×10\hat{ }5 centimeters/second, and fc is the transmitted frequency.

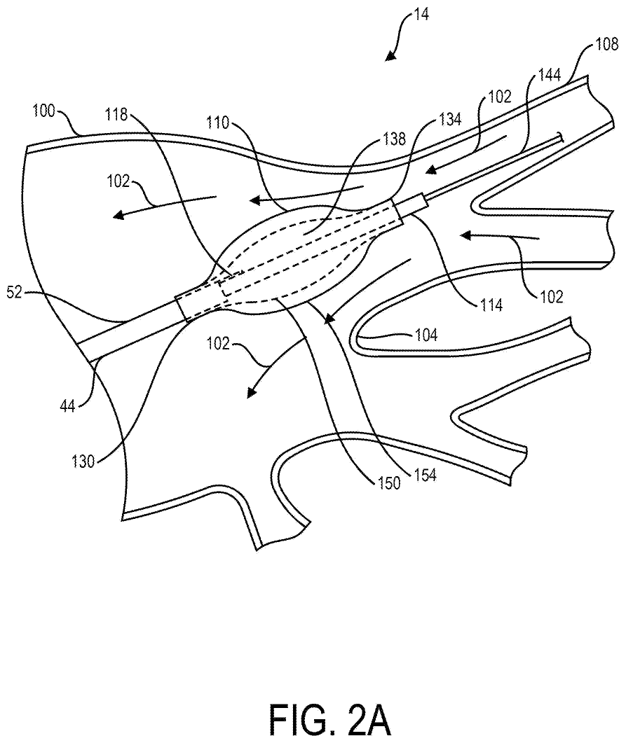

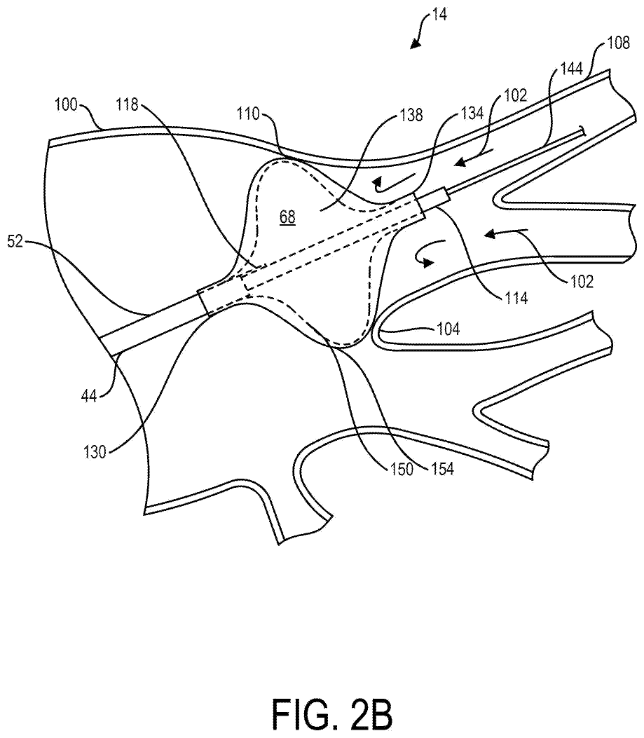

The cryogenic balloon catheter system 10 then determines whether the vein has been occluded, the degree or level of occlusion of the vein, and/or whether ablation may proceed based on the multiple velocity measurements, as described below in relation to FIGS. 2A and 2B.

FIGS. 2A and 2B are diagrams illustrating the balloon catheter 14 partially inflated or deflated in a pulmonary vein 108 and the balloon catheter 14 inflated to occlude the pulmonary vein 108, according to embodiments of the disclosure.

More specifically, FIG. 2A is a simplified schematic view illustration of a portion of the patient 12 and a portion of the cryogenic balloon catheter system 10 with an expandable balloon 110 that is partially inflated or deflated in the pulmonary vein 108 and blood flow 102 through the pulmonary vein 108 and past the expandable balloon 110, according to embodiments of the disclosure. FIG. 2B is a simplified schematic view illustration of a portion of the patient 12 and a portion of the cryogenic balloon catheter system 10 with the expandable balloon 110 inflated to occlude the pulmonary vein 108, such that blood flow 102 past the expandable balloon 100 is prevented, according to embodiments of the disclosure.

FIGS. 2A and 2B are schematic illustrations of the distal end portion 52 of the balloon catheter 14 positioned within a selected anatomical region of the patient 12, in this case, a left atrium 100 adjacent to an ostium 104 of a pulmonary vein 108, such as when the system 10 is used in a pulmonary vein isolation (PVI) procedure to terminate an atrial fibrillation. The balloon catheter 14 includes the expandable balloon 110, a guidewire lumen 114 and an injection tube 118. As shown, the balloon 110 has a proximal end 130 and an opposite distal end 134 and defines an internal space 138 that creates a cryo-chamber during a cryoablation procedure. In embodiments, the proximal end 130 of the balloon 110 is attached to the distal end portion 52 of the shaft 44, and the distal end 134 of the balloon 110 is attached to the guidewire lumen 114 near the distal end thereof. Also, in embodiments, the injection tube 118 is disposed within and extends from the shaft 44, and terminates within and is open to the internal space 138. The injection tube 118 is operable to deliver the cryogenic fluid 68 to the internal space 138.

Although not shown in FIGS. 2A and 2B, the balloon catheter 14 also includes an exhaust lumen within the shaft 44 and open to the internal space 138. The exhaust lumen is operable to facilitate evacuation of the cryogenic fluid 68 from the internal space 138 and to facilitate inflation of the balloon 110. In embodiments, the guidewire lumen 114 may be slidable relative to the shaft 44 to facilitate expansion and subsequent collapse of the balloon 110 in use.

As illustrated, an instrument 144 is shown extending through and beyond the guidewire lumen and into the pulmonary vein 108. As the skilled artisan will appreciate, the instrument 144 may be a guidewire, mapping wire or catheter, anchoring wire, or other medical device useful to facilitate the a cryo-therapy procedure. However, the use of the instrument 144 is optional.

In the embodiments of FIGS. 2A and 2B, the balloon 110 is a dual-balloon construction including an inner balloon 150 and an outer balloon 154. The balloons 150 and 154 are configured such that the inner balloon 150 receives the cryogenic fluid 68 and the outer balloon 154 surrounds the inner balloon 150. The outer balloon 154 acts as part of a safety system to capture the cryogenic fluid 68 in the event of a leak from the inner balloon 150. It is understood that the balloon catheter 14 can include other structures as well. However, for the sake of clarity, these other structures have been omitted from the figures. Additionally, it is further appreciated that in some alternative embodiments, the balloon catheter 14 includes only a single balloon.

The balloon catheter 14 is positioned within the left atrium 100 of the patient 12. The guidewire 144 and guidewire lumen 114 are inserted into a pulmonary vein 108 of the patient 12, and the catheter shaft 44 and the balloons 150 and 154 are moved along the guidewire 144 and/or the guidewire lumen 114 to be positioned near the ostium 104 of the pulmonary vein 108.

During use, the inner balloon 150 can be partially or fully inflated so that at least a portion of the inner balloon 150 expands against at least a portion of the outer balloon 154. Once the inner balloon 150 is sufficiently inflated, an outer surface of the outer balloon 154 can then be positioned to abut and/or substantially form a seal with the ostium 104 of the pulmonary vein 108 to be treated.

The inner balloon 150 and the outer balloon 154 can be formed from any suitable materials. For example, in some embodiments, the inner balloon 150 can be formed from a sturdy material to better inhibit leaks of the cryogenic fluid 68 that is received therein, and the outer balloon 154 can be made from a relatively compliant material to ensure better contact and positioning between the outer balloon 154 and the pulmonary vein 108.

During balloon cryoablation procedures, prior to delivering the cryo-ablative energy, the operator can inflate the balloon 110 using the cryogenic fluid 68 at a relatively high temperature, i.e., above the temperature used to ablate the target tissue. In this way, the operator can achieve balloon-tissue contact and vein occlusion to increase probability of vein isolation before starting the ablation. In addition, to minimize procedure time, it can be desirable to utilize the exhaust lumen of the balloon catheter 14 as a conduit for delivering the cryogenic fluid 68 to the internal space 138 during the inflation phase, i.e., due to its relatively large size compared to the injection tube 118. It is also desirable to maintain relatively close control over the inflation pressure during the cryoablation procedure. For example, a drop in the inflation pressure can result in partial deflation of the balloon 110 and consequent or diminishment of balloon-tissue contact and vessel occlusion.

As will be appreciated, proper vein occlusion is an important factor in accomplishing pulmonary vein isolation via cryoablation. In particular, inadequate occlusion can result in blood flow past the surface of the balloon 110, reducing the efficiency of heat transfer between the target tissue and the balloon 110, which in turn can increase ablation procedure time or, in some cases, inhibit the formation of an ablation lesion capable of creating the desired conduction block. To verify vein occlusion and achieve good vein occlusion, the cryogenic balloon catheter system 10 determines whether the vein has been occluded, the degree or level of occlusion of the vein, and/or whether ablation may proceed, based on multiple velocity measurements of the blood flow 102.

In embodiments, the cryogenic balloon catheter system 10 determines the overall change in velocity of the blood flow 102 in the vein 108 from before inflation of the balloon 110 to after inflation of the balloon 110. In embodiments, the cryogenic balloon catheter system 10 determines changes in velocity of the blood flow 102 during inflation of the balloon 110.

In embodiments, V₀ is the velocity of the blood flow 102 in the pulmonary vein 108 before inflation of the balloon 110 in the pulmonary vein 108, such as depicted in FIG. 2A, Vₙ is the velocity of the blood flow 102 in the pulmonary vein 108 after inflation of the balloon 110 in the pulmonary vein 108, such as depicted in FIG. 2B, and the change of velocity is given by the following Formula 2:

ΔV=V₀−Vₙ Formula 2

where, if there is good occlusion of the pulmonary vein 108, Vₙ≈0, and ΔV is higher and closer to the value of V₀.

In another aspect, the cryogenic balloon catheter system 10 monitors the change in the velocity ΔV and stabilization of the change in the velocity ΔV over time. In embodiments, the cryogenic balloon catheter system 10 determines the velocity or speed of the blood flow 102 in the pulmonary vein 108 on a periodic basis, such as once every 40 milliseconds (ms), and calculates the change in the velocity ΔV for each sample. Over a longer periodic interval, such as 2 seconds, the samples of the changes in the velocity ΔV are averaged to obtain an average change in velocity over the longer periodic interval. The average change of the change in velocity values can be monitored over time to determine whether the vein, such as the pulmonary vein 108, has been occluded, the degree or level of occlusion of the vein, and/or whether ablation may proceed

For example, determining the velocity every 40 ms over a 2 second interval results in 50 samples of the change in velocity taken over the 2 second interval. These 50 samples are averaged to provide an average change in velocity value over the 2 second interval, as indicated in the following Formula 3:

$${Δ}_{average} = {\sum }_{k = 1}^{50} \frac{Δ {V}_{k} - Δ {V}_{k - 1}}{50} \qquad Formula 3$$

Better occlusion is indicated where: ΔV approaches V₀, which means Vₙ≈0; and Δ_{average} is zero or approaches zero. In embodiments, ΔV is compared to a corresponding threshold value, which may be determined using V₀ or a percentage of V₀, and the level of occlusion is determined and/or a go/no-go indication is provided for the ablation procedure. In embodiments, Δ_{average} is compared to a corresponding stability threshold value and the degree of occlusion is determined or a go/no-go indication is provided for the ablation procedure.

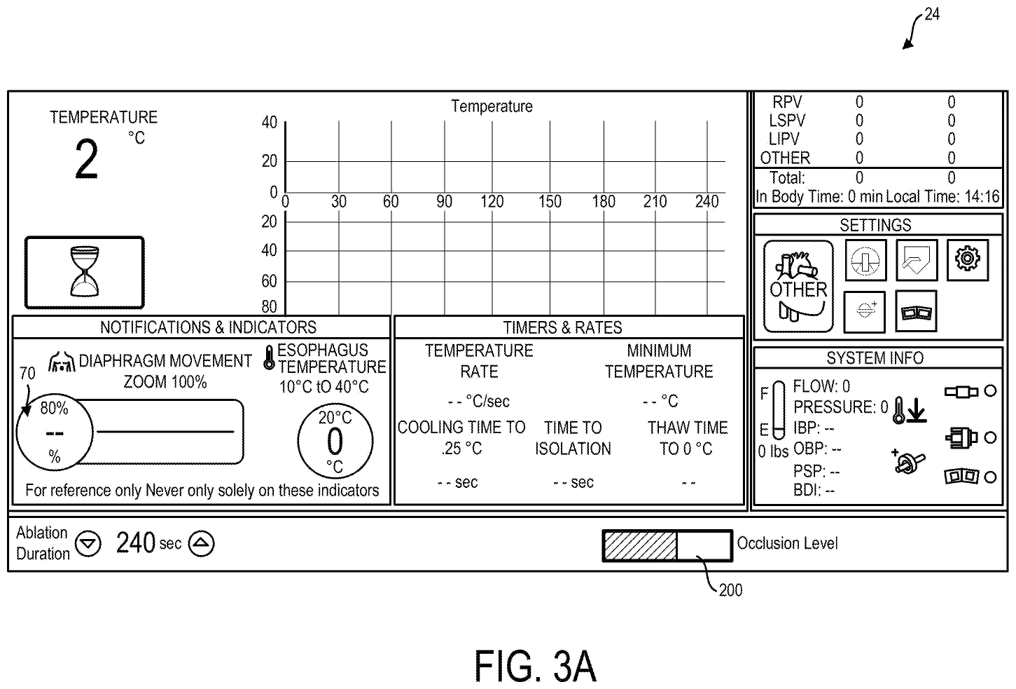

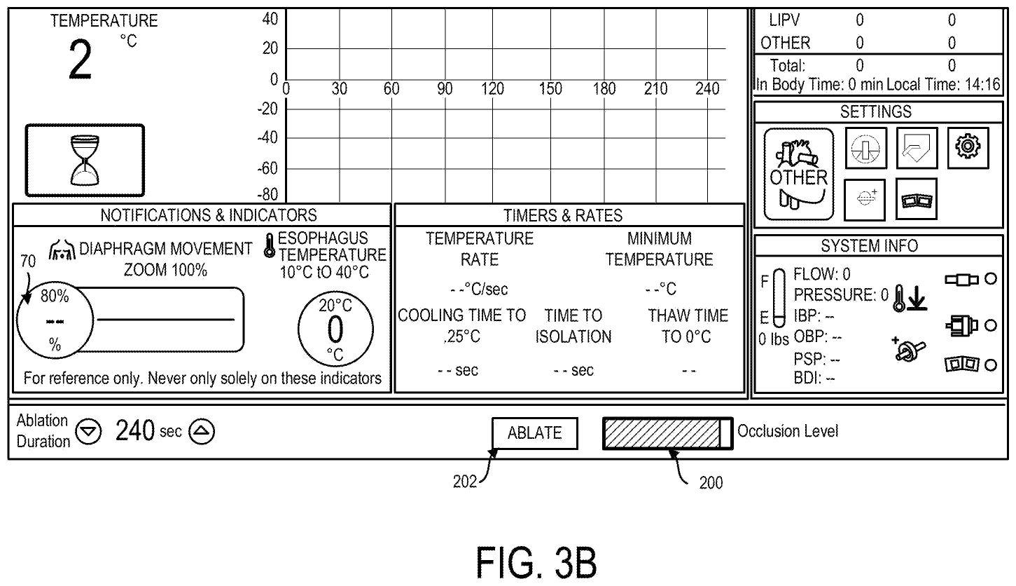

FIGS. 3A and 3B are diagrams illustrating the graphical display 24 during an ablation procedure including a vein isolation procedure, according to embodiments of the disclosure.

FIG. 3A is a diagram illustrating the graphical display 24 including an occlusion level indicator 200 prior to reaching a satisfactory level of occlusion for ablation to proceed, according to embodiments of the disclosure. The occlusion level indicator 200 is a dynamic level indicator that changes over time during the ablation and the vein isolation procedure. Also, the graphical display 24 includes various frames and other representations 70 to provide static and dynamic visual data and information to the operator.

In embodiments, as the balloon 110 is inflated in the pulmonary vein 108, the occlusion level indicated in the occlusion level indicator 200 increases from a smaller bar width to a larger bar width corresponding to increases in the overall change in the velocity of the blood flow ΔV and decreases in the Δ_{average}. In embodiments, the occlusion level indicator 200 indicates the level of occlusion in color, such as red to yellow bar widths prior to reaching a satisfactory level of occlusion for ablation to proceed and green bar widths after reaching a level of occlusion where ablation may proceed.

FIG. 3B is a diagram illustrating the graphical display 24 including the occlusion level indicator 200 and an ablation button 202 after reaching a level of occlusion where ablation may proceed, according to embodiments of the disclosure. The occlusion level indicator 200 turns green and the ablation button 202 lights up, i.e., is displayed, after reaching the level of occlusion where ablation may proceed. In embodiments, the level of occlusion where ablation may proceed is determined using one or more threshold values for the overall change in the velocity of the blood flow ΔV and the Δ_{average} levels.

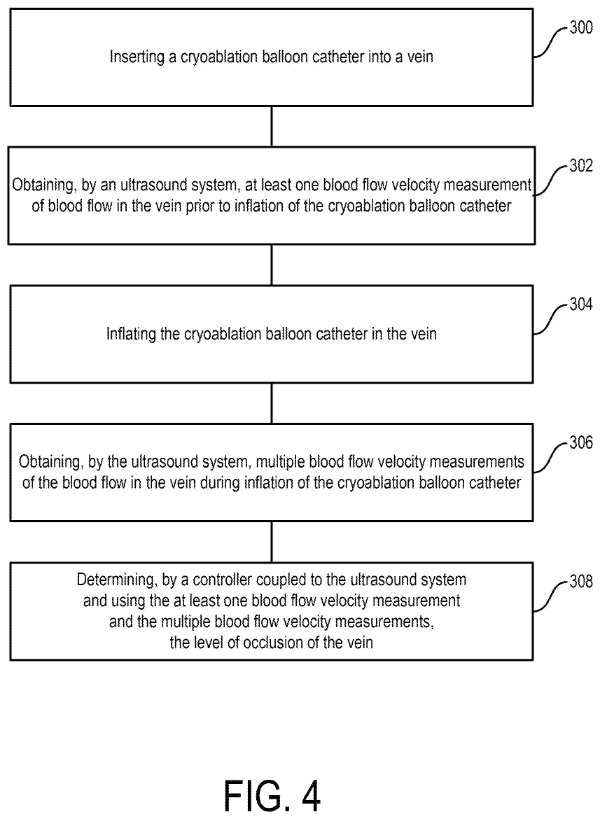

FIG. 4 is a flow chart diagram illustrating a method of determining a level of occlusion of a vein and/or whether the vein has been occluded, according to embodiments of the disclosure.

At 300, the method includes inserting a cryoablation balloon catheter into a vein. In embodiments, this includes inserting a cryoablation balloon catheter, such as cryoablation balloon catheter 14, into a vein, such as the pulmonary vein 108. Also, in some embodiments, this includes inserting a cryoablation balloon catheter into an ostium of the vein.

Next, at 302, the method includes obtaining, by an ultrasound system, at least one blood flow velocity measurement of blood flow in the vein prior to inflation of the cryoablation balloon catheter. In embodiments, this includes obtaining a measure of V₀ which is the velocity of the blood flow 102 in the pulmonary vein 108 before inflation of the balloon 110 in the pulmonary vein 108, such as depicted in FIG. 2A.

At 304, the method includes inflating the cryoablation balloon catheter in the vein. Which, in embodiments, includes inflating the cryoablation balloon catheter 14 in the ostium 104 of the pulmonary vein 108, such as depicted in FIG. 2B.

At 306, the method includes obtaining, by the ultrasound system, multiple blood flow velocity measurements of the blood flow in the vein during inflation of the cryoablation balloon catheter.

And, at 308 the method includes determining, by a controller coupled to the ultrasound system and using the at least one blood flow velocity measurement and the multiple blood flow velocity measurements, the level of occlusion of the vein and/or whether the vein has been occluded.

In embodiments, determining the level of occlusion of the vein includes determining changes of blood flow velocity in the vein and determining average changes in the changes of the blood flow velocity during multiple time periods. Also, in embodiments, determining the level of occlusion of the vein includes comparing one or more of the average changes to a threshold value, such as a stability threshold value.

In addition, in embodiments, determining the level of occlusion of the vein includes determining an overall change of blood flow velocity in the vein from prior to inflation of the cryoablation balloon catheter to after inflation of the cryoablation balloon catheter and, in embodiments, comparing the overall change of the blood flow velocity in the vein to an overall threshold value.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

What is claimed is:

1. A control system for a cryogenic ablation system, the control system comprising:
an extracorporeal ultrasound sensor configured to detect blood flow in a vein and generate an output signal indicative of blood flow velocity of the blood flow in the vein; and
a controller configured to receive the output signal and determine whether the vein has been occluded by a cryoablation balloon catheter.

2. The system of claim 1, wherein the controller is configured to determine changes in the blood flow velocity and to determine average changes in the changes in the blood flow velocity during multiple time periods to determine whether the vein has been occluded.

3. The system of claim 2, wherein the controller compares one or more of the average changes to a stability threshold value to determine whether the vein has been occluded.

4. The system of claim 1, wherein the controller determines an overall change in the blood flow velocity to determine whether the vein has been occluded.

5. The system of claim 4, wherein the controller compares the overall change in the blood flow velocity to an overall change threshold value to determine whether the vein has been occluded.

6. The system of claim 1, wherein the vein is a pulmonary vein.

7. The system of claim 1, wherein the controller is an ultrasound controller and the extracorporeal ultrasound sensor is coupled to the ultrasound controller and configured to transmit and receive ultrasound frequencies through patient tissues, wherein the ultrasound controller is configured to determine the blood flow velocity.

8. The system of claim 1, wherein the controller is configured to determine the blood flow velocity based on a doppler frequency shift in the blood flow.

9. The system of claim 1, wherein the controller prevents ablation from proceeding until the controller has determined that the vein has been occluded.

10. The system of claim 1, wherein the controller provides one or more of an audio and a visual alarm in response to loss of occlusion of the vein.

11. A cryogenic ablation system for determining whether a pulmonary vein has been occluded, the system comprising:
a cryoablation balloon catheter configured to be inserted into an ostium of the pulmonary vein;
an extracorporeal ultrasound sensor configured to detect blood flow in the pulmonary vein before, during, and after inflation of the cryoablation balloon catheter in the ostium of the pulmonary vein and to generate output signals indicative of blood flow velocity of the blood flow in the pulmonary vein; and
a controller coupled to the extracorporeal ultrasound sensor and configured to receive the output signals and determine blood flow velocity values to determine whether the pulmonary vein has been occluded by the cryoablation balloon catheter.

12. The system of claim 11, wherein the controller is configured to inflate the cryoablation balloon catheter in response to manually pushing an inflate control button.

13. The system of claim 11, wherein the controller determines at least one of average changes in changes of the blood flow velocity values during multiple time periods and an overall change in the blood flow velocity values from prior to inflation of the cryoablation balloon catheter to after inflation of the cryoablation balloon catheter.

14. The system of claim 11, further comprising a display, wherein the controller is configured to visually indicate on the display an occlusion level of the pulmonary vein.

15. The system of claim 11, further comprising a display, wherein the controller is configured to visually indicate on the display that ablation may proceed.

16. A method of determining a level of occlusion of a vein, the method comprising;
inserting a cryoablation balloon catheter into an ostium of the vein;
obtaining, by an ultrasound system, at least one blood flow velocity measurement of blood flow in the vein prior to inflation of the cryoablation balloon catheter;
inflating the cryoablation balloon catheter in the ostium of the vein;
obtaining, by the ultrasound system, multiple blood flow velocity measurements of the blood flow in the vein during inflation of the cryoablation balloon catheter; and
determining, by a controller coupled to the ultrasound system and using the at least one blood flow velocity measurement and the multiple blood flow velocity measurements, the level of occlusion of the vein.

17. The method of claim 16, wherein determining the level of occlusion of the vein includes determining changes of blood flow velocity in the vein and determining average changes in the changes of the blood flow velocity during multiple time periods.

18. The method of claim 17, wherein determining the level of occlusion of the vein includes comparing one or more of the average changes to a threshold value.

19. The method of claim 16, wherein determining the level of occlusion of the vein includes determining an overall change of blood flow velocity in the vein from prior to inflation of the cryoablation balloon catheter to after inflation of the cryoablation balloon catheter.

20. The method of claim 19, wherein determining the level of occlusion of the vein includes comparing the overall change of the blood flow velocity in the vein to a threshold value.
